(19) United States
(12) Patent Application Publication
(10) Pub. No.: US 2006/0169794 A1
(43) Pub. Date: Aug. 3, 2006

(54) Perfume dispenser

(76) Inventor: Kwok Keung Yeung, Hong Kong (CN)

Correspondence Address: Raymond Y. Chan, Suite 128, 108 N. Ynez Ave., Monterey Park, CA, 91754, US

(21) Appl. No.: 11/394,864

(22) Filed: Mar. 31, 2006

Related U.S. Application Data
(62) Division of application No. 10/759,739, filed on Jan. 16, 2004.

(30) Foreign Application Priority Data
Jan. 18, 2003 (CN) 03223125.3
Jan. 18, 2003 (CN) 03223124.5
Jul. 24, 2003 (CN) 03268066.X

(51) Int. Cl. A61L 9/04

(52) U.S. Cl. 239/44

(57) ABSTRACT

A perfume dispenser includes a squeezable bag and a dispensing element provided at the squeezing bag. The squeezable bag has a storage chamber for containing a predetermined volume of perfume agent and a guiding channel communicatively extended from the storage chamber. The dispensing element is communicating with the storage chamber through the guiding channel, wherein the dispensing element is adapted for absorbing the perfume agent from storage chamber through the guiding channel when a compressing force is applied on the squeezable bag so as to release the perfume agent to outside.

### CROSS-REFERENCE OF RELATED APPLICATION

This is a divisional application of a non-provisional application having an application Ser. No. 10/759,739 and a filing date of Jan. 16, 2004.

### FIELD OF INVENTIONS

The present invention relates to a perfume container, and more particularly to a perfume dispenser adapted for sealedly containing a perfume agent in a squeezable bag to provide a long lasting and convenient service.

### BACKGROUND OF PRESENT INVENTION

Fragrance is highly desirable in our daily lives. As a result, perfumes, due to its odor releasing character, have been widely used by people in different occasions. The odor of perfume could be sticky around the user and last for a while, thus enriching the user's impressive image as well as bringing user a refreshed feeling. Moreover, a good perfume could adjust user's emotion, since fragrances released from perfume could prettify user's elegant demeanor so as to enhance user's self-confidence. In addition, perfume's effect is not merely limited to cosmetic application. In reality, perfume could be applied in a wide variety of purposes, for instance, perfume could refresh user's mind or keep user calm, and some perfumes could be utilized as disinfectant, disease preventative means or health care products. Nevertheless, when applied directly onto user's skin, perfume has been failed showing a sustained effect. This is due to the fact that perfume is a sort of volatilized substance having an effective time limit around 1-2 hours after human skin practice. Therefore, how to prolong perfume's effecting period have drawn remarkable attentions for years in the arts.

Meanwhile, the disposed plastic bag is widely used worldwide thanks to its cheaper cost, one-time-using convenience, and hygiene. As a result, a lot of liquid products, including perfume, were packaged by plastic bag in routine application. Ordinarily, a plastic liquid packaging bag is air-tight sealed or tightened. After liquid is poured into the plastic packaging bag, a bag sealer is provided to prevent liquid of packaging bag from seeping out. Here, thermoplastic sealer is used in general application. Once the user wants to use the liquid, she has to cut an opening on the plastic packaging bag to discharging liquid to a container for later use, for example pouring the perfume liquid into a bottle, etc. In other words, currently used plastic bags are suitable only for packaging and delivery not for convenient practical usages. i.e., the user has to pour out the liquid of plastic bag to hard material containers for aftermath practices. Therefore, providing a plastic liquid bag by which users could control seeping out or pouring out procedures will undoubtedly win overwhelming respects in packaging industry. This is due to the fact that a plastic bag having controllable seeping or flowing out function will enormously widen the plastic bag's range of application.

### SUMMARY OF PRESENT INVENTION

A main object of present invention is to provide a perfume dispenser, wherein a perfume agent is sealedly contained in a squeezable bag to release through a dispensing element such that the perfume agent is adapted for seeping and flowing in a controllable manner.

Another object of present invention is to provide a perfume dispenser wherein a casing is provided to receive the squeezable bag, and a simple dispensing arrangement is incorporated with the casing for urging perfume agent of the squeezable bag flow out.

Another object of present invention is to provide a perfume dispenser which could continuously release odor to skin or surrounding air.

Another object of the present invention is to provide an adjustable perfume dispenser which could be attached to various ornaments to solve abovementioned problems.

Another object of present invention is to provide a perfume dispenser wherein no expensive parts or complicated structures involved.

Another object of present invention is to provide a perfume dispenser which is convenient and clean.

Accordingly, in order to accomplish the above objects, the present invention provides a perfume dispenser, comprising:
a squeezable bag having a storage chamber for containing a predetermined volume of perfume agent and a guiding channel communicatively extended from the storage chamber; and
a dispensing element provided at the squeezable bag to communicate with the storage chamber through the guiding channel, wherein the dispensing element is adapted for absorbing the perfume agent from storage chamber through the guiding channel when a compressing force is applied on the squeezable bag so as to release the perfume agent to outside. In addition, the guiding channel can be formed as at least one S-shaped conduit to prolong the distance between the storage chamber and the dispensing element for prohibiting the perfume agent from flowing out disorderly.

For convenience, the squeezable bag could be placed into a dispensing device which comprises a casing for receiving the squeezable bag and a dispensing actuation arrangement having an actuating member moveably mounted to the casing of the dispensing device, a driving member slidably disposed next to the squeezable bag placed in the casing of the dispensing device to apply a compressing force at the squeezable bag, and a resilient member disposed there between the actuating member and the driving member to provide a sustained urging force on the driving member. The actuating member could be adjusted to force driving member to apply a compressing force at the squeezable bag causing liquid of perfume agent in the squeezable bag flow out through guiding channel, and then to be absorbed into dispensing element to release perfume odor. Meanwhile, an opening is provided on the casing for purposely opening and closing the perfume dispenser.

These and other objectives, features, and advantages of the present invention will become apparent from the following detailed description, the accompanying drawings, and the appended claims

### BRIEF DESCRIPTION OF THE DRAWINGS

FIG. 1 is a sectional view of perfume dispenser according to the preferred embodiment of present invention illustrating the squeezable bag attached on a soft nature membrane.

FIG. 2 is a sectional view of perfume dispenser according to the preferred embodiment of present invention illustrating the squeezable bag being attached on a wrist watch band.

FIG. 3 is a sectional view of the perfume dispense of an alternative mode according to the preferred embodiment of present invention showing the S-shaped guiding channel embedded in squeezable bag.

FIG. 4 is a sectional view of the perfume dispense of an alternative mode according to the preferred embodiment of present invention showing two S-shaped guiding channels embedded in squeezable bag and a dispensing element attached on the squeezable bag.

FIG. 5 is a perspective view of perfume dispenser according to the preferred embodiment of present invention showing the casing in which the squeezable bag is disposed.

FIG. 6 is an exploded perspective view of perfume dispenser according to the preferred embodiment of present invention showing the casing as well as the dispensing actuation arrangement comprising a driving member and an actuating member.

FIG. 7 is a perspective view of perfume dispenser of another alternative mode according to the preferred embodiment of present invention illustrating the squeezable bag disposed in a curve-shaped casing.

FIG. 8 is a perspective view of perfume dispenser of another alternative mode according to the preferred embodiment of present invention illustrating the squeezable bag disposed in a curved-column shaped casing.

FIG. 9 is perspective view of perfume dispenser of another alternative mode according to the preferred embodiment of present invention illustrating the squeezable bag disposed in a button-shaped casing.

FIG. 10 is an exploded perspective view of button-shaped casing in FIG. 9 showing a pushing member, a resilient member and a threaded cross-shaped actuating member.

### DETAILED DESCRIPTION OF THE PREFERRED EMBODIMENT

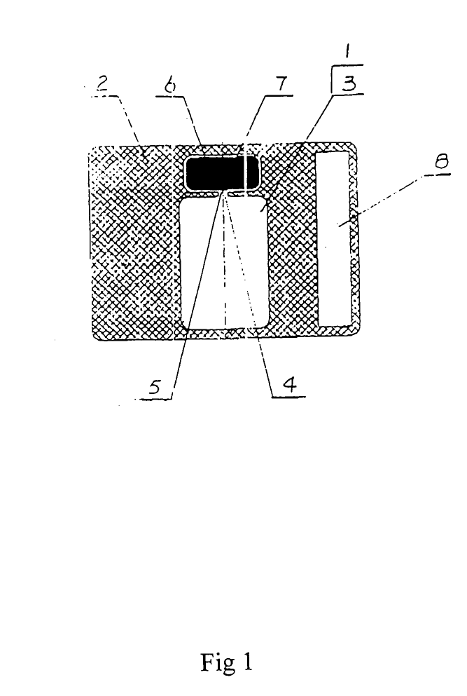

Referring to FIG. 1, a perfume dispenser according to the preferred embodiment of present invention is illustrated, wherein the perfume dispenser comprises a squeezable bag 1 and a dispensing element 7. The squeezable bag 1, having a portable size, comprises a storage chamber 3 for containing a predetermined volume of perfume agent, and a guiding channel 4 communicatively extended from the squeezable bag 1.

The dispensing element 7 is provided at the squeezable bag 1 to communicate with the storage chamber 3 to outside through the guiding channel 4, wherein the dispensing element 7 is adapted for absorbing the perfume agent from the storage chamber 3 through the guiding channel 4 once a compressing force is applied on the squeezable bag 1 so as to gradually and continuously release the perfume agent through the dispensing element 7 to outside.

According to the preferred embodiment, the squeezable bag 1 is provided at the middle portion of a rectangular-shaped soft membrane 2. The squeezable bag 1 further has a sealing chamber 6, having a releasing opening, is disposed at an outlet 5 of the guiding channel 4 wherein a dispensing element 7 is provided in the sealing chamber 6 for absorbing the perfume agent from storage chamber 3 through guiding channel 4 such that once a compressing force is applied on the squeezable bag 1, the perfume agent is then gradually and continuously releasing through the dispensing element 7 to outside.

A sticking element 8 is provided on one side of the soft membrane 2 to attach the squeezable bag 1 of perfume dispenser to an object surface, such as clothing. Accordingly, the sticking element 8 could be a sticky sticking layer or a magic stick.

Here, the squeezable bag 1 could be a soft nature plastic bag for one time disposable use, wherein the dispensing element 7 is made of spongy material adapted for absorbing the perfume agent and for gradually releasing the perfume agent to the surroundings.

To use the perfume dispenser according to the preferred embodiment of present invention, the user merely carries the perfume dispenser on the user's body via a wrist watch band. Accordingly, the perfume dispenser is firstly wrapped around the wrist watch band such that the user is able to securely mount the perfume dispenser to the wrist watch band via the sticking element 8. Therefore, the user wears the wrist watch band to carry the perfume dispenser while the perfume agent is adapted to be discharged through the releasing opening on the sealing chamber 6. Preferably, the dispensing element 7 is a spongy layer having a distinctive liquid absorbing ability. In addition, the releasing opening of the sealing chamber 6 can be formed by pricking off the sealing chamber 6 by a needle or any sharp means to expose the dispensing element 7 to outside. Under normal circumstance, no perfume agent (may be extremely small amount of perfume agent) would seep out or flow out unless the compressing force was applied on the squeezable bag 1.

Once the user presses against the squeezable bag 1, the perfume agent in squeezable bag 1 is forced to flow out through the guiding channel 4 and guide into sealing chamber 6 through the outlet 5 of the guiding channel 4. Then, the perfume agent will be immediately absorbed by the dispensing element 7 provided in the sealing chamber 6. Ultimately, the liquid of perfume agent immersed, and gradually, impregnated in the dispensing element 7 will be smeared on user's skin or clothes so as to releasing the odor. After the perfume agent immersed in the dispensing element 7 is thoroughly vaporized, the user repeatedly presses the squeezable bag 1 again to force liquid of perfume agent to flow into the sealing chamber 6 and to be absorbed by dispensing element 7, as a consequence, the perfume agent immersed into dispensing element 7 will be smeared onto user's skin or clothes to continuously release odor until liquid of squeezable bag 1 is fully drained out. Therefore, the perfume dispenser of the present invention is good for one time disposable use while being cost effective.

Under normal circumstance, the guiding channel 4 is sealed since there is no pressure difference between the storage chamber 3 of squeezable bag 1 and the exterior thereof, and the perfume agent within squeezable bag 1 is kept in a balance manner. However, once the compressing force is applied on the squeezable bag 1, the pressure of squeezable bag 1 would have been changed thus forcing the perfume agent in the squeezable bag 1 to flow out through guiding channel 4.

In other words, the user could control the seepage as well as dispensing of the perfume agent by applying pressure on the squeezable bag 1. It is worth to mention that no perfume agent (may be extremely small amount of perfume agent) would have been discharged unless the user applies pressure on the squeezable bag 1. Since the sticking 5 element 8 is provided on the soft membrane 2, the squeezable bag 1 could be easily and effectively attached to other objects, such as clothes or ornaments of users, thereafter providing users of huge convenience.

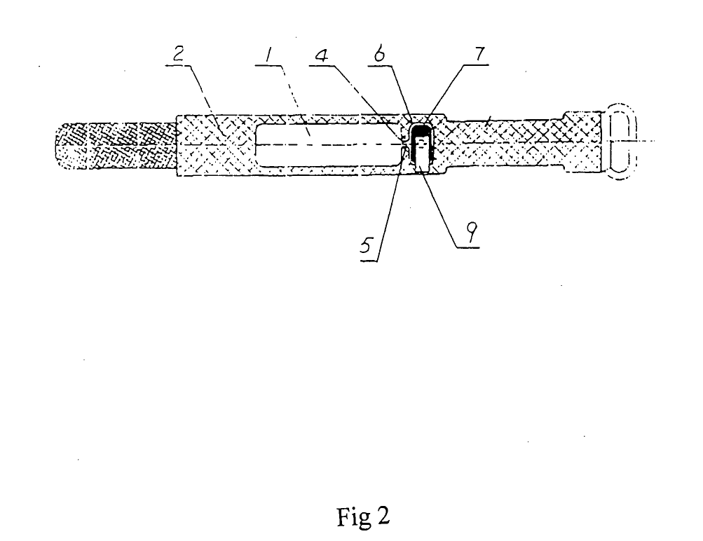

Referring to FIG. 2, a sectional view of perfume dispenser according to the preferred embodiment of present invention is illustrated showing the squeezable bag 1 being attached with a wrist watch band. Compared with structure shown in FIG. 1, the basic structure of perfume dispenser is almost the same; the only difference is that squeezable bag 1 is formed as a strip-shape for being fittingly attached to the face of wrist watch band.

For convenience, a detachable sealer 9 is detachably attached at the releasing opening of the sealing chamber 6 for controlling the perfume agent from being released therethrough. In other words, the user could tear off the detachable sealer 9 for exposing the dispensing element 7 disposed in sealing chamber 6 through the releasing opening thereof to release perfume agent to outside. Accordingly, the releasing opening can be pre-formed on the sealing chamber 6 such that once the detachable sealer 9 is detached from the sealing chamber 6 to expose the releasing opening, the perfume agent is adapted to be slowly discharged therethrough.

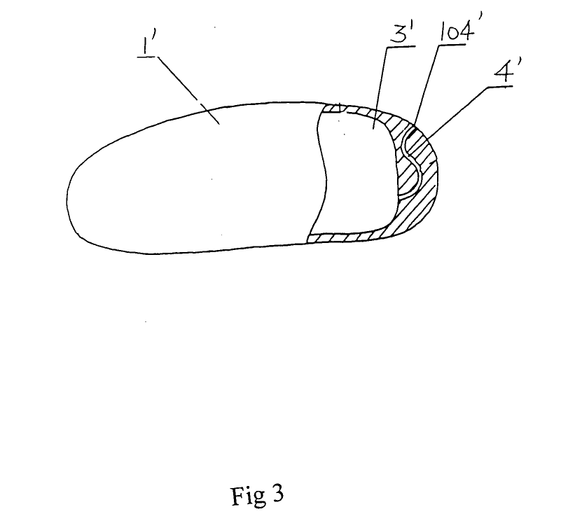

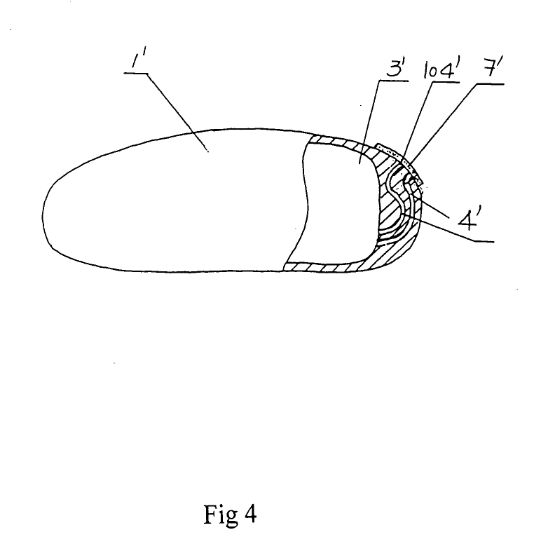

Referring to FIG. 3 and FIG. 4, an alternative mode of perfume dispenser according to the preferred embodiment of present invention is illustrated. The squeezable bag 1′, having a portable size, comprises a storage chamber 3′ for containing a predetermined volume of perfume agent, and a guiding channel 4′ communicatively extended from the squeezable bag 1′.

The dispensing element 7′ is provided at the squeezable bag 1′ to communicate with the storage chamber 3′ to outside through the guiding channel 4′, wherein the dispensing element 7′ is adapted for absorbing the perfume agent from the storage chamber 3′ through the guiding channel 4′ once a compressing force is applied on the squeezable bag 1′ so as to gradually and continuously release the perfume agent through the dispensing element 7′ to outside.

As shown in FIGS. 3, and 4, the guiding channel 4′ is shaped as an S-shaped conduit extended from the storage chamber 3′ to the dispensing element 7′ so as to prolong a distance between the storage chamber 3′ and the dispensing element 7′ for guiding a flow of the perfume agent towards the dispensing element 7′ in a controlling manner. Accordingly, one end of the guiding channel 4′ is communicatively connected to the storage chamber 3′ of squeezable bag 1′, while the opposite end of the guiding channel 4′ is extended to outside forming a discharging aperture 104′. It is worth to mention that by prolonging the guiding channel 4′, the perfume agent in squeezable bag 1′ will be prohibited from flowing out disorderly through discharging aperture 104′. In addition, two S-shaped guiding channels 4′ could be provided in the squeezable bag 1′ to promptly replenish perfume agent vaporized in the dispensing element 7′, as shown in FIG. 4.

The dispensing element 7′ is provided on an outer surface of the squeezable bag 1′ to cover the discharging aperture 104′ of guiding channel 4′ for absorbing perfume agent through the guiding channel 4′. Once the compressing force is applied on the squeezable bag 1′, the perfume agent in the storage chamber 3′ is forced to flow out through the guiding channel 4′, and then, be immediately absorbed by dispensing element 7′ provided at squeezable bag 1 through the discharging aperture 104′. Finally, the perfume agent immersed in dispensing element 7′ will release odor to outside. It is worth to mention that since the dispensing element 7′ is made of sponge material and is provided on the outer surface of the squeezable bag 1′, the user is able to directly apply the perfume agent on the user's skin by rubbing the dispensing element 7′ thereon.

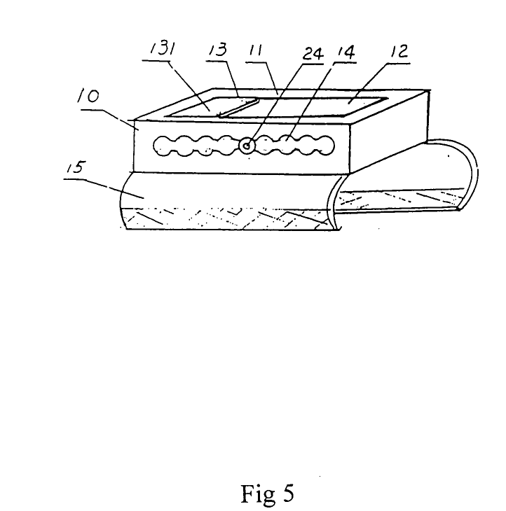

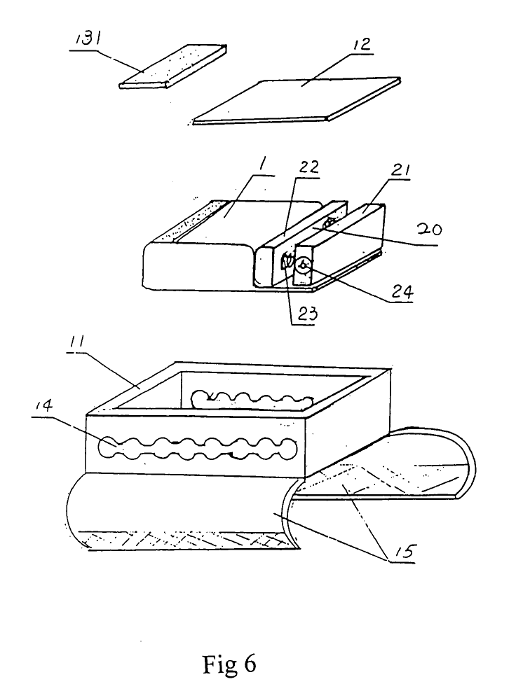

Referring to FIG. 5 and FIG. 6, the perfume dispenser, according to the preferred embodiment of present invention, further comprises a dispensing device 10 wherein the squeezable bag 1 is placed in a dispensing device 10.

The dispensing device 10 comprises a casing 11 replaceably receiving the squeezable bag 1 therein and a dispensing actuation arrangement 20 comprising a driving member 22 slidably disposed in said casing and an actuating member 21 movably engaged with the casing 11 to drive the driving member 22 towards the squeezable bag 1 for applying the compressing force on the squeezable bag 1 within the casing 11. The dispensing device 10 further comprises a carrying device 15 provided on the casing 11 for attaching the casing 2 on an object.

The casing 11, having a box shaped, has a releasing outlet 13, a sealing cover 12 covering on the casing 11 to enclose the squeezable bag 1 therein and a sliding cover 131 slidably mounted on the casing 11 to communicate the squeezable bag 1 with outside through the releasing outlet 13.

The casing 11 has at least an elongated engaging slot 14 provided on a sidewall of the casing 11, wherein an end portion of the actuating member 21 is transversely and slidably engaged with the engaging slot 14 in such a manner that when the actuating member 21 is slid within the casing 11 along the engaging slot 14, the driving member 22 is pushed to slide within the casing 11 to press on the squeezable bag 1 at the storage chamber 3 for releasing the perfume agent to the dispensing element 7 so as to dispense the perfume agent out of the casing 11.

Accordingly, two engaging slots 14 are provided on two sidewalls of casing 11 respectively to guide the sliding movement of the actuating member 21 to push the driving member 22 towards the squeezable bag 1 within the casing 11 so as to apply the compressing force on squeezable bag 1 to release perfume agent.

As shown in FIGS. 5 and 6, the dispensing actuation arrangement 20 further comprises a locker member 24 provided at the end portion of the actuating member 21 to lock up the end portion of the actuating member 21 at the respective sidewall of the casing 11 so as to retain the compressing force in a controlling manner.

Accordingly, the engaging slot 14 is shaped to form a plurality of locking portions aligning along the sidewall of the casing 11, wherein the locker member 24 is slidably mounted at the end portion of the actuating member 21 to engage with one of the locking portions of the engaging slot 14 to retain the actuating member 21 in position.

In order to unlock the actuating member 21, the locker member 24 is slidably pressed to disengage with the respective locking portion of the engaging slot 14 such that the actuating member 21 is allowed to transversely slide within the casing 11 along the engaging slot 14 so as to move forward to the squeezable bag 1. It is worth to mention that when the locker member 24 is engaged with the adjacent locking portion of the engaging slot 14, the driving member 22 is pushed further to the squeezable bag 1 to release the perfume agent.

In addition, to apply the perfume bag 3 with a sustained compressing force, a resilient member 23 is disposed in the casing 11, wherein the resilient member 23, which is a compression spring, has two ends biasing against the actuating member 21 and the driving member 22 respectively in such a manner that when the actuating member 21 is slid within the casing 11, the resilient member 23 applies an urging force to push the driving member 22 towards the squeezable bag 1. Meanwhile, the resilient member 23 could sustain a continuous urging force applied on squeezable bag 1 to keeping perfume agent of squeezable bag 1 seep out slowly and gradually to release odor while the actuating member 21 is restricted in a locked position that the locker member 24 is engaged with the engaging slot 14.

To use this perfume dispenser, the user needs to open the releasing outlet 13 by sliding a sliding cover 131 and slide the actuation member 21 to a side of the casing 11 for reserving a room to place the squeezable bag 1. Afterwards, the squeezable bag 1 is placed into casing 11 wherein the outlet of guiding channel 4 of squeezable bag 1 is aligned to face towards the releasing outlet 13 of the casing 11. Once the sliding cover 131 cover the releasing outlet 13 of the casing 11, the squeezable bag 1 is entirely closed in the casing 11 and is prohibited from releasing odor to outside as well. On the other hand, if the user wishes to release odor, she merely needs to slidably open the sealing cover 131 to expose the dispensing element 7 to outside through the releasing outlet 13.

If the odor released from the casing 10 is faint, she could adjust the dispensing actuation arrangement 20 by shifting the actuating member 21 to force the driving member 22 to press squeezable bag 1 thus causing perfume agent of squeezable bag 1 seep out and to be absorbed into the dispensing element 7 to release perfume odor. It is worth to mention that perfume agent of squeezable bag 1 will not continuously discharge unless the dispensing actuation arrangement 20 could sustain a compressing pressure to the squeezable bag 1, while liquid of perfume agent absorbed into the dispensing element 7 could last for a while to release odor until the perfume agent immersed into the dispensing element 7 fully vaporized. In other words, after the odor released from dispensing element 7 volatized faint smell, the user could unlock the locker member 24 such that the actuating member 21 is adapted to shift to push the driving member 22 towards the squeezable bag 1 so as to refill the perfume agent on the dispensing element 7. Meanwhile, it is worth to mention that the carrying device 15, which is formed as a bracelet, mounted on casing 11 to hold the casing 11 on the user's arm.

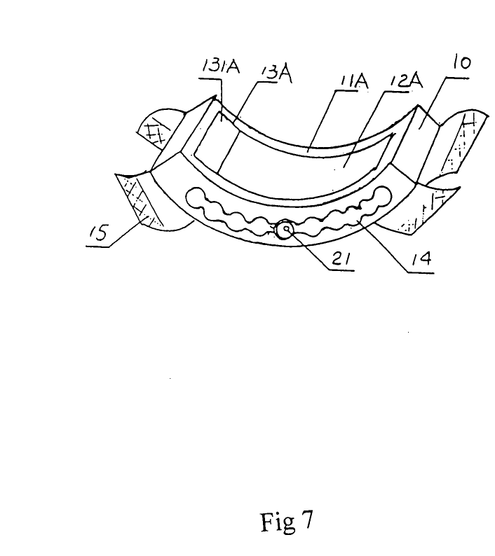

FIG. 7 illustrates an alternative mode of the casing 11A showing the casing 11A is shaped in an arc-shaped member to receive the squeezable bag 1 therein. Here, the casing 11A, in which the squeezable bag 1 is disposed, the sliding cover 131A and the sealing cover 13A are all designed into curved structure to form the releasing outlet 13A having a curved shaped. This is due to the reason for better attaching present perfume dispenser to ring-shaped ornament such as bracelet or wrist watch band to form an appearance as a whole.

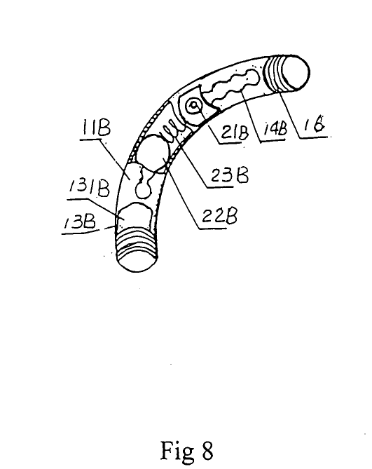

Referring to FIG. 8, another alternative mode of the casing 11B according to the preferred embodiment of present invention is illustrated to show the squeezable bag 1 disposed in a tubular ring-shaped casing 11B to integrate with a bracelet.

The casing 11B has a releasing outlet 13B and a sliding cover 131B slidably mounted on the casing 11B to enclose the releasing outlet 13B. Accordingly, the curved elongate engaging slot 14B is provided on the sidewall of casing 11B wherein the actuating member 21B is slidably mounted in the casing 11B for forcing the driving member 22B having a spherical shaped to apply a compressing force to squeezable bag 1. And two threaded ends 16 are provide on two end portions of casing 10B to engage with bracelet. In addition, a resilient member 23B is disposed in the casing 11B wherein the resilient member 23B has two ends biasing against the actuating member 21B and the spherical driving member 22B respectively in such a manner that when the actuating member 21B is slid within the casing 11B, the resilient member 23B applies an urging force to push the driving member 22B towards the squeezable bag 1. Meanwhile, the resilient member 23B could sustain a continuous urging force applied on squeezable bag 1 to continuously and gradually release perfume agent while the actuating member 21B is restricted in a locked position. Here, the tubular ring-shaped casing 11B could be applied as a portion of ring-shaped bracelet to forming as an integrated structure and appearance, since this sort of perfume dispenser not only preserve bracelet's esthetical appearance, but also continuously release perfume odor to outside.

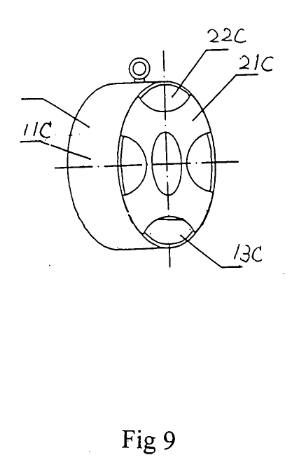

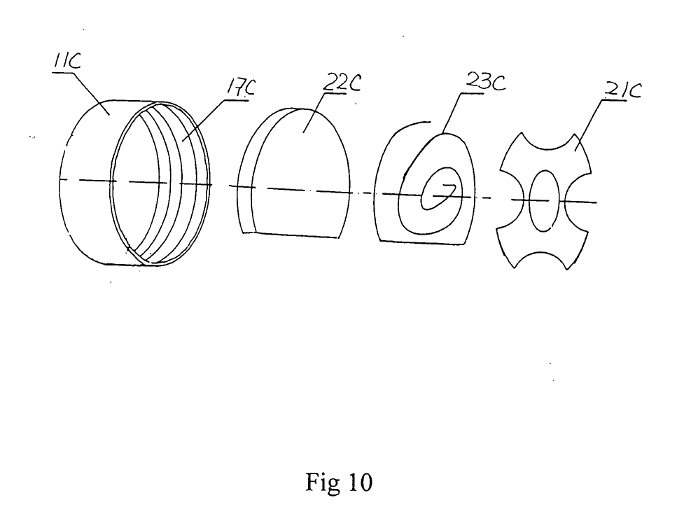

Referring to FIG. 9 and FIG. 10, another alternative mode according to the preferred embodiment of present invention is illustrated showing the squeezable bag 1 being disposed in the casing 11C.

The driving member 22C is a disc shaped body disposed in the casing 11C to sandwich the squeezable bag 1 between the driving member 22C and a bottom wall of the casing 11C and defines a side opening to exposed the dispensing element 7 in the casing 11C, wherein the actuating member 21C forms as a casing cover rotatably engaging with the casing 11C not only to enclose the squeezable bag 1 within the casing 11C but also to drive the driving member 22C to press against the storage chamber 3 of the squeezable bag 1 for releasing the perfume agent therein.

Accordingly, the casing 11C has a thread portion 17C formed on an inner wall wherein the actuating member 21C is rotatably engaged with the thread portion 17C of the casing 11C to selectively adjust a depth of the casing 11C. In other words, the actuating member 21C is rotated to push the driving member 22C towards the bottom wall of the casing 11C so as to press against the squeezable bag 1.

In addition, n resilient member 23C, which is a coil spring, is disposed within the casing 11C having two ends biasing against the driving member 22C and the actuating member 21C respectively in such a manner that when the actuating member 21C is rotated within the casing 11C, the resilient element 23C applies an urging force to push the driving member 22C to rotate so as to press against the squeezable bag 1.

After the squeezable bag 1 is placed in casing 11C, the driving member 22C, the resilient member 23C are disposed respectively to press the squeezable bag 1 wherein the side opening of the driving member 22C is purposely placed to align with the dispensing element 7 on squeezable bag 1 to release perfume agent. The actuating member 21C is rotated to engage with the thread portion 17C of the casing 11C while the resilient element 23C is sandwiched between the actuating member 21C and the driving member 22C. Thereafter, by rotating the actuating member 21C, the resilient element 23C transforms the rotating force of the actuating member 21C into a linear movement to push the driving member 22C towards the bottom wall of the casing 11C. At the same time, the resilient member 23C applies the compressing force on the squeezable bag 1 via the driving member 22C thus forcing the perfume agent of squeezable bag 1 to seep out through guiding channel 4 and to be absorbed into dispensing element 7 for releasing perfume odor to outside. Meanwhile, by rotating the actuating member 21C from time to time with respect to casing 11C, the user could control the compressing force applied to the squeezable bag 1 to guarantee liquid of squeezable bag 1 seeping out continuously until liquid of perfume agent in squeezable bag 1 is drained out.

Conclusively, the perfume dispenser of present invention is rational in structure, exquisite, small in volume, and convenient. It could be made into an independent structure, such as a small container or attached to other objects such as user's clothes or ornaments according to user's intention. For instances, the perfume dispenser could be bound with the wrist watch strip, be part of bracelet, or be exposed directly to user's skin, or just could be directly used as a clamping appliance, or ultimately integrated with ornaments. According to the present invention, no liquid could be released under normal circumstance. Therefore, no contamination and wasting issues would have been involved. Finally, the perfume dispenser is easy to use, inexpensive to make, convenient and clean.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A perfume dispenser, comprising:
a squeezable bag, having a portable size, having a storage chamber for containing a predetermined volume of perfume agent, and a guiding channel communicatively extended from said storage chamber;
a dispensing element provided at said squeezable bag to communicate said storage chamber to outside through said guiding channel, wherein said dispensing element is adapted for absorbing said perfume agent from said storage chamber through said guiding channel once a compressing force is applied on said squeezable bag so as to gradually and continuously release said perfume agent through said dispensing element to outside; and
a dispensing device which comprises:
a casing having a tubular ring-shape integrated with a bracelet for a user to wear, wherein said squeezable bag is received in said casing;
a dispensing actuation arrangement comprising a driving member slidably disposed in said casing and an actuating member movably engaged with said casing to drive said driving member towards said squeezable bag for applying said compressing force on said squeezable bag within said casing.

2. The perfume dispenser, as recited in claim 1, wherein said casing has at least an elongated engaging slot provided on a sidewall of said casing, wherein an end portion of said actuating member is slidably engaged with said engaging slot in such a manner that when said actuating member is slid within said casing along said engaging slot, said driving member is pushed to slide within said casing to press on said squeezable bag at said storage chamber for releasing said perfume agent to said dispensing element.

3. The perfume dispenser, as recited in claim 1, wherein said dispensing actuation arrangement further comprises a resilient element disposed in said casing, wherein said resilient element has two ends biasing against said actuating member and said driving member respectively in such a manner that when said actuating member is slid within said casing, said resilient element applies an urging force to push said driving member towards said squeezable bag.

4. The perfume dispenser, as recited in claim 2, wherein said dispensing actuation arrangement further comprises a resilient element disposed in said casing, wherein said resilient element has two ends biasing against said actuating member and said driving member respectively in such a manner that when said actuating member is slid within said casing, said resilient element applies an urging force to push said driving member towards said squeezable bag.

5. The perfume dispenser, as recited in claim 1, wherein said driving member has a ball shape movably disposed in said casing to press against said squeezable bag.

6. The perfume dispenser, as recited in claim 2, wherein said driving member has a ball shape movably disposed in said casing to press against said squeezable bag.

7. The perfume dispenser, as recited in claim 4, wherein said driving member has a ball shape movably disposed in said casing to press against said squeezable bag.

8. The perfume dispenser, as recited in claim 1, wherein said casing further has a releasing outlet and comprises a sealing cover which is slidably mounted on said casing at said releasing outlet and is arranged in such a manner that when said sealing cover is slid to expose said releasing outlet, said dispensing element is aligned with said releasing outlet for releasing odor therefrom, and when said sealing cover is slid to cover said releasing outlet, said squeezable bag is enclosed in said casing for preventing said odor releasing from said dispensing element to an exterior of said casing through said releasing outlet.

9. The perfume dispenser, as recited in claim 4, wherein said casing further has a releasing outlet and comprises a sealing cover which is slidably mounted on said casing at said releasing outlet and is arranged in such a manner that when said sealing cover is slid to expose said releasing outlet, said dispensing element is aligned with said releasing outlet for releasing odor therefrom, and when said sealing cover is slid to cover said releasing outlet, said squeezable bag is enclosed in said casing for preventing said odor releasing from said dispensing element to an exterior of said casing through said releasing outlet.

10. The perfume dispenser, as recited in claim 7, wherein said casing further has a releasing outlet and comprises a sealing cover which is slidably mounted on said casing at said releasing outlet and is arranged in such a manner that when said sealing cover is slid to expose said releasing outlet, said dispensing element is aligned with said releasing outlet for releasing odor therefrom, and when said sealing cover is slid to cover said releasing outlet, said squeezable bag is enclosed in said casing for preventing said odor releasing from said dispensing element to an exterior of said casing through said releasing outlet.

11. The perfume dispenser, as recited in claim 7, wherein said casing has two threaded ends engaging with said bracelet to form a ring shaped ornament.

12. The perfume dispenser, as recited in claim 10, wherein said casing has two threaded ends engaging with said bracelet to form a ring shaped ornament.

13. The perfume dispenser, as recited in claim 11, wherein said squeezable bag further has a sealing chamber, having a releasing opening, communicating said storage chamber through said guiding channel, wherein said dispensing element is disposed in said sealing chamber for releasing said perfume agent from said storage chamber to outside through said releasing opening.

14. The perfume dispenser, as recited in claim 12, wherein said squeezable bag further has a sealing chamber, having a releasing opening, communicating said storage chamber through said guiding channel, wherein said dispensing element is disposed in said sealing chamber for releasing said perfume agent from said storage chamber to outside through said releasing opening.

15. The perfume dispenser, as recited in claim 13, further comprising a detachable sealer detachably attached at said releasing opening of said sealing chamber for controlling said perfume agent from being released therethrough.

16. The perfume dispenser, as recited in claim 14, further comprising a detachable sealer detachably attached at said releasing opening of said sealing chamber for controlling said perfume agent from being released therethrough.

17. The perfume dispenser, as recited in claim 15, wherein said guiding channel is shaped as a S-shaped conduit extended from said storage chamber to said dispensing element so as to prolong a distance between said storage chamber and said dispensing element for guiding a flow of said perfume agent towards said dispensing element in a controlling manner.

18. The perfume dispenser, as recited in claim 16, wherein said guiding channel is shaped as a S-shaped conduit extended from said storage chamber to said dispensing element so as to prolong a distance between said storage chamber and said dispensing element for guiding a flow of said perfume agent towards said dispensing element orderly in a controlling manner.

19. The perfume dispenser, as recited in claim 17, wherein said dispensing element is made of sponge material adapted for absorbing said perfume agent and for gradually releasing said perfume agent.

20. The perfume dispenser, as recited in claim 18, wherein said dispensing element is made of sponge material adapted for absorbing said perfume agent and for gradually releasing said perfume agent.
